(19) United States
(12) Patent Application Publication
(10) Pub. No.: US 2017/0196446 A1
(43) Pub. Date: Jul. 13, 2017

(54) Contact Lens Assessment

(71) Applicant: IRISS Medical Technologies Limited, London (GB)

(72) Inventors: Yuval Yashiv, London (GB); Ron Uriel Maor, London (GB); Nigel Andrew Simon Barnard, London (GB)

(73) Assignee: IRISS Medical Technologies Limited, London (GB)

(21) Appl. No.: 15/313,524

(22) PCT Filed: May 22, 2015
(86) PCT No.: PCT/EP2015/061462

§ 371 Date: Nov. 22, 2016

(30) Foreign Application Priority Data
May 24, 2014 (GB) 1409291.0

(51) Int. Cl. A61B 3/00; G06K 9/00; A61B 3/11; A61B 3/14; A61B 3/10

(52) U.S. Cl. CPC A61B 3/0025; A61B 3/14; A61B 3/1005; A61B 3/112; G06K 9/0061

(57) ABSTRACT

A method and apparatus (100) for processing an image to determine one or more parameters of a contact lens suitable for a human or animal subject. The image comprises a plurality of pixels forming an image of at least part of one eye of the subject. The apparatus comprises: an iris detector (116) configured to determine a diameter of an iris of the subject; a cornea determiner (118) configured to determine a diameter of a cornea of the subject based on the determined diameter of the iris; and a contact lens determiner (120) configured to determine one or more parameters of contact lens for the subject based on the determined cornea diameter.

### TECHNICAL FIELD

The invention relates to methods and apparatus for determining one or more parameters of a contact lens for a subject. In some exemplary embodiments, the parameters may comprise physical and/or optical parameters of a contact lens. Further, in exemplary embodiments, the invention may also relate to, but is not limited to, specific parameters such as the size of the contact lens and the position of optical features in a contact lens.

### BACKGROUND

Most contact lenses fitted today are chosen from a range of off-the-shelf models. These models differ mostly in their refractive power, so the main consideration when fitting them is the patient's prescription for eyeglasses.

Rejection rates—people who stop using their contact lenses—are typically very high and are about 50% in some studies. Of these, some are due to external factors such as dry or sensitive eyes, but about half simply report that they do not see as well with the contacts as they would like. Made-to-order lenses, which would take into account the size of the cornea, may help reduce the prevalence of this issue.

Multifocal lenses, which are always made-to-order, require accurate measurement of eye features for optimal fitting. These measurements are often carried out roughly, using a ruler, or not at all.

### SUMMARY

According to the invention in a first aspect, there is provided an apparatus for processing an image to determine one or more parameters of a contact lens suitable for a human or animal subject, the image comprising a plurality of pixels forming an image of at least part of one eye of the subject, the apparatus comprising: an iris detector configured to determine a diameter of an iris of the subject; a cornea determiner configured to determine a diameter of a cornea of the subject based on the determined diameter of the iris; and a contact lens determiner configured to determine the one or more parameters of a contact lens for the subject based on the determined cornea diameter.

Optionally, the apparatus further comprises a camera configured to capture the image and a light source associated with the camera.

Optionally, the apparatus further comprises an eye detector configured to locate one or more eyes of the subject in the image.

Optionally, the eye detector is configured to identify in the image reflections of a light source used when the image was captured, based on the RGB values of pixels of the image, and to identify which of the reflections is from the at least one eye of the subject.

Optionally, the eye detector is configured to identify which of the reflections is from the at least one eye of the subject based on one or more of: the shape of the reflection; the size of the reflection; the RGB values of the reflection; and, in images comprising two eyes, a substantially horizontal distance between a pair of reflections.

Optionally, the iris detector is configured to determine the diameter of the iris by determining an iris ellipse having a best fit to the iris of the subject.

Optionally, the iris detector is configured to determine iris pixels of the image based on a luma value of each pixel in an eye region, and to determine the iris ellipse as an ellipse of a plurality of ellipses having the most iris pixels inside.

Optionally, the iris pixels have a luma value less than a threshold value.

Optionally, the iris detector is configured to determine the iris ellipse based on one or more of a luma value for iris pixels, a saturation value for the iris pixels and a redness value for the iris pixels.

Optionally, the iris detector is configured to determine the redness of a pixel based on the greater of the red value minus the green value or the red value minus the blue value for the pixel.

Optionally, the iris detector is configured to classify pixels proximal to the iris as “pink”, “white” or “other” and to determine a score for each of a plurality of possible iris ellipses based on:

NWO−4*NWI+NOI−NOO

where NWI is the number of white pixels inside the ellipse, NWO is the number of white pixels outside the ellipse, NOI is the number of other pixels inside the ellipse and NOO is the number of other pixels outside the ellipse,
and wherein the iris detector is further configured to determine the iris ellipse as the ellipse having the highest score.

Optionally, the iris detector is configured to determine a horizontal visible iris diameter, HVID, as the horizontal diameter of the iris ellipse.

Optionally, the iris detector is configured to determine one or more of: a vertical iris diameter; and a diagonal iris diameter, based on the iris ellipse.

Optionally, the contact lens determiner is configured to determine the one or more parameters of the contact lens based on the vertical iris diameter and/or the diagonal iris diameter.

Optionally, the iris detector is configured to determine an approximate centre of the iris.

Optionally, the iris detector is configured to determine the approximate centre of the iris by determining an approximate horizontal centre line of the iris by determining horizontal maxima of the iris pixels.

Optionally, the one or more parameters of the contact lens comprise physical parameters and/or optical parameters of the contact lens.

Optionally, the contact lens determiner is configured to determine a diameter of a contact lens based on the cornea diameter.

Optionally, the apparatus further comprises a pupil detector configured to determine a diameter and/or an eccentricity of a pupil of the subject, and wherein the contact lens determiner is configured to determine the one or more parameters of the contact lens based on the determined pupil diameter and/or pupil eccentricity.

Optionally, the contact lens determiner is configured to determine a location of one or more optical features of a contact lens based on a determined pupil eccentricity.

Optionally, the pupil detector is configured to determine the diameter of the pupil by determining a pupil ellipse that has a best fit with the pupil of the subject.

Optionally, the pupil ellipse is determined based on a luma value of pixels in the eye region.

Optionally, the pupil detector is configured to classify the pixels in the eye region as pupil pixels having a luma value less than a threshold value and iris pixels having a luma value greater than a threshold value, and to determine a score for each of a plurality of possible pupil ellipses based on:

PDI−PDO

where PDI is the proportion of pupil pixels inside the ellipse and PDO is the proportion of pupil pixels outside the ellipse, and wherein the pupil detector is further configured to determine the pupil ellipse as the ellipse having the highest score.

Optionally, the pupil detector is configured to determine the pupil ellipse based on a brightness ignoring red, BIR, value of pixels in the eye region.

Optionally, the pupil detector is configured to classify the pixels in the eye region as pupil pixels having a BIR value less than a threshold value and iris pixels having a BIR value greater than a threshold value, and to determine a score for each of a plurality of possible pupil ellipses based on:

PDI−PDO

where PDI is the proportion of pupil pixels inside the ellipse and PDO is the proportion of pupil pixels outside the ellipse, and wherein the pupil detector is further configured to determine the pupil ellipse as the ellipse having the highest score.

Optionally, the pupil detector is configured to determine a centre of the pupil, the apparatus further comprising a lid margin detector configured to determine a distance from the centre of the pupil to one or more eyelids of the subject.

Optionally, the contact lens determiner is configured to determine one or more optical parameters of the contact lens based on the determined distance from the centre of the pupil to one or more eyelids, and wherein the optical parameter comprises a location of optical features on the contact lens.

According to the invention in a further aspect, there is provided a method for processing an image to determine one or more parameters of a contact lens suitable for a human or animal subject, the image comprising a plurality of pixels forming an image of at least part of one eye of the subject, the method comprising: determining, by an iris detector, a diameter of an iris of the subject; determining, by a cornea determiner, a diameter of a cornea of the subject based on the determined diameter of the iris; and determining, by a contact lens determiner, the one or more parameters of a contact lens for the subject based on the determined cornea diameter.

As is clear from the description below, further method steps and/or optional features of the method corresponding to the above mentioned features of the apparatus are possible.

According to the invention in a third aspect, there is provided a computer program comprising instructions which, when executed on at least one processor, cause the at least one processor to carry out any method set out herein.

According to the invention in a fourth aspect, there is provided a carrier containing the computer program set out above, wherein the carrier is one of an electronic signal, optical signal, radio signal, or non-transitory computer readable storage medium.

### BRIEF DESCRIPTION OF THE DRAWINGS

Exemplary embodiments of the invention are described herein with reference to the accompanying drawings, in which:

FIG. 1 is a schematic representation of an apparatus for determining a size of a contact lens suitable for a human or animal subject;

FIG. 2 is a flow diagram showing a method for determining a size of a contact lens suitable for a human or animal subject;

FIG. 3 is a schematic representation of an image of an eye with horizontal strips superimposed thereon;

FIG. 4 is a schematic representation of an image of an eye with a plurality of possible iris ellipses superimposed thereon;

FIG. 5 is a schematic representation of an image of an eye with a bounding box superimposed thereon;

FIG. 6 is a schematic representation of an image of an eye showing various features thereof;

FIG. 7 is a schematic representation of an image of an eye showing corneal curvature; and

FIG. 8 is a schematic representation of an image of an eye showing the lid margins and pupil eccentricity.

### DETAILED DESCRIPTION

The inventors have appreciated that a major reason for rejection rates in the take up of contact lenses is that the contact lenses provided are incorrectly sized or have certain parameters, such as optical features, incorrectly positioned. Specifically, the inventors have appreciated that accurate determination of the size of the cornea and/or other eye features, of a subject can lead to better fitting contact lenses and lower rejection rates.

Human corneas vary within a size range of over 30% and off-the-shelf contact lenses are made to fit the most common, median value of this range, which is typically about 11.4 mm. A larger or smaller cornea than the median value may result in poor fitting of a contact lens and in poor vision through a contact lens.

Measuring the cornea directly is difficult and the HVID (horizontal visible iris diameter) may be used as a proxy for the cornea. HVID is typically about 0.4 mm larger than the cornea, and it may be directly observed as the horizontal ‘white-to-white’ distance from one side of the iris to the other.

In exemplary methods and apparatus disclosed herein, HVID may be used as a first screening step to determine one or more parameters of a contact lens, such as diameter or base curve, for a particular subject. This allows a determination of which subjects are unsuitable for off-the-shelf contact lenses, thereby reducing rejection rates. In practice, HVID is often not measured. When it is measured, this is typically done by holding a ruler to the eye of a subject or using a topographer, which measures the distance directly. This method is clearly inaccurate and the differences between a “typical” HVID (about 11.8 mm) and a “small” or “large” HVID are approximately 1-2 mm, which is difficult to see with a ruler.

It is noted that methods and apparatus disclosed herein relate to any type of contact lens, such as soft or hard lenses, scleral lenses, corneal lenses, multifocal lenses and toric lenses. Further, it is noted that, as used herein, the term “optical features” of a lens encompasses those features that are configured to correct the vision of a wearer. The optical features may be lens features configured to bend light according to a particular eye condition. It is noted that the term “contact lens” relates to the device to be fitted to the eye and not all of the contact lens actually comprises optical features.

Generally, disclosed herein are methods and apparatus for processing an image comprising an eye of a subject to determine a size of the iris. The size of the iris is used to determine the size of the cornea, which in turn is used to determine one or more parameters of a contact lens size for the subject. The determined contact lens parameters may be used to determine whether the subject is suitable for off the shelf contact lenses and/or for manufacturing made to order contact lenses.

The contact lens parameters may comprise physical parameters and/or optical parameters. Physical contact lens parameters include the diameter of the contact lens and the base curve of the contact lens. The term “base curve” is a well known term of art and refers to a radius of curvature of an inner surface of a contact lens. Optical contact lens parameters include the optical features of the contact lens, such as the features that affect the optical power of the contact lens. The positioning of the optical contact lens parameters may be particularly important when optical features are non-uniform across the contact lens, for example toric contact lenses or multifocal contact lenses.

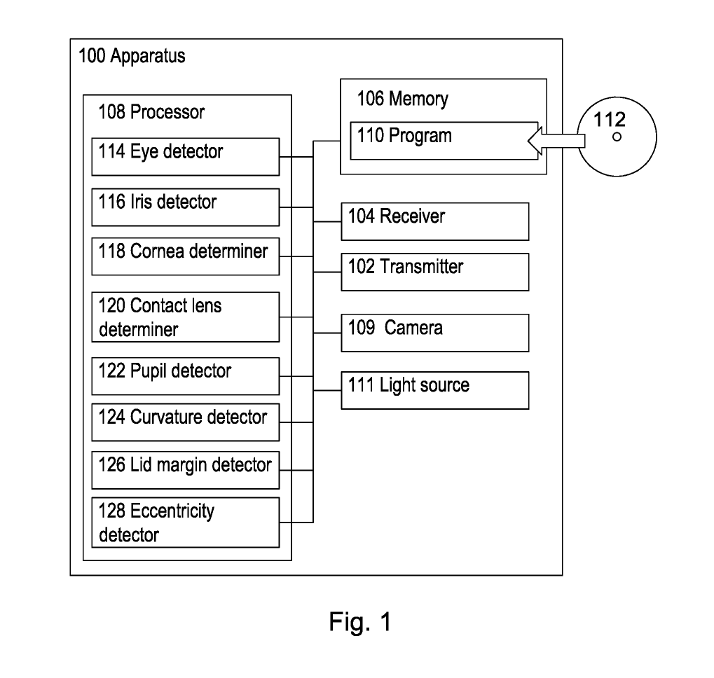

FIG. 1 shows an apparatus 100 for determining one or more parameters of a contact lens of a subject. The apparatus may be a computer device, such as a personal computer (PC), laptop computer, smartphone or other computing device.

The apparatus 100 may comprise a transmitter 102 and a receiver 104. The transmitter 102 and receiver 104 are in electrical communication with other apparatus or devices for example over a telecommunications network or a wired or wireless connection and are configured to transmit and receive data accordingly.

It is noted that the term “electrical communication” encompasses both wired and wireless electrical communication. Therefore, electrical communication may be, for example, a network communication over a wired connection or a network communication of over a radio frequency connection.

The apparatus 100 further comprises a memory 106 and a processor 108. The memory 106 may comprise a non-volatile memory and/or a volatile memory. The memory 106 may have a computer program 110 stored therein. The computer program 110 may be configured to undertake the methods disclosed herein. The computer program 110 may be loaded in the memory 106 from a non-transitory computer readable medium 112, on which the computer program is stored. The processor 108 is configured to undertake at least the functions of an eye detector 114, an iris detector 116, a cornea determiner 118, a contact lens determiner 120, a pupil detector 122, a curvature detector 124, a lid margin detector 126 and an eccentricity detector 128, as set out herein.

The apparatus 100 may also comprise an image capturing means, such as a camera 109. The camera 109 is configured to capture images comprising at least part of one or more eyes of a human or animal subject. Images captured by the camera 109 may be stored in the memory 106 for later processing. The apparatus 100 may also include a light source 111 associated with the camera 109 to illuminate the subject as images are captured.

Each of the transmitter 102 and receiver 104, memory 106, processor 108, camera 109, light source 111, eye detector 114, iris detector 116, cornea determiner 118, contact lens determiner 120, pupil detector 122, curvature detector 124, lid margin detector 126 and eccentricity detector 128 is in electrical communication with the other features of the apparatus 100. The apparatus 100 can be implemented as a combination of computer hardware and software. In particular, the eye detector 114, iris detector 116, cornea determiner 118, contact lens determiner 120, pupil detector 122, curvature detector 124, lid margin detector 126 and eccentricity detector 128 may be implemented as software configured to run on the processor 108. The memory 106 stores the various programs/executable files that are implemented by a processor 108, and also provide a storage unit for any required data. The programs/executable files stored in the memory 106, and implemented by the processor 108, can include an eye detector 114, iris detector 116, cornea determiner 118, contact lens determiner 120, pupil detector 122, curvature detector 124, lid margin detector 126 and eccentricity detector 128, but are not limited to such.

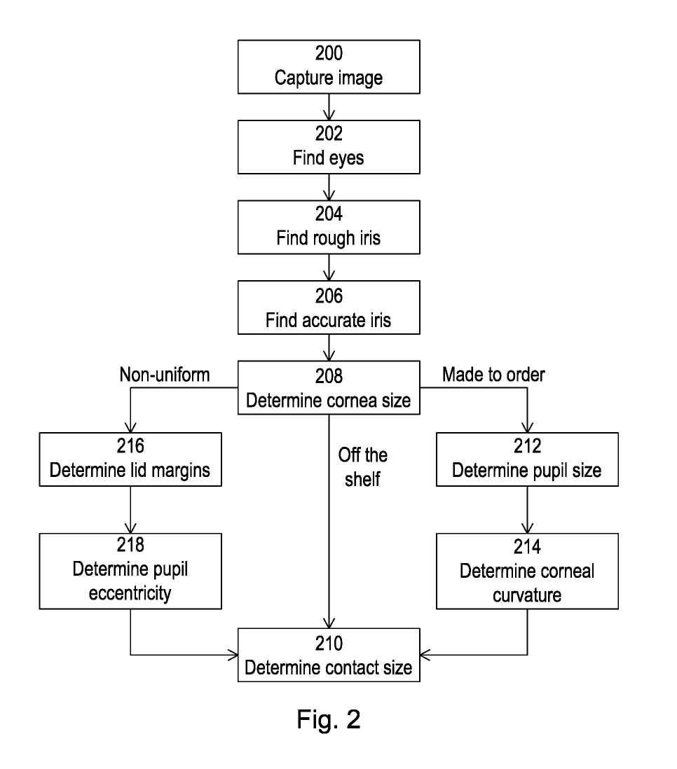

FIG. 2 shows an exemplary method for determining one or more physical parameters of contact lens for a subject.

An image is captured 200 by the camera 109. The captured image is stored in the memory 106. This step may be omitted in exemplary methods as the image may be captured by a separate device and passed to the apparatus 100 for processing.

In exemplary methods and apparatus, the image should conform to the following rules:
Both eyes should be visible together in the captured image on a substantially horizontal line;
The picture should be taken at a range of about 50 cm;
The face should fill the photo so typically optical zoom is needed;
Resolution of the image should be high enough to give a ratio of at least 20 pixels per mm;
The distance at which the picture is taken is known, typically provided by a focus mechanism of the camera 109;
The camera provides a flash or other source of light which reflects off of the eyes of the subject.

Although the above example specifies that both eyes should be visible together in the captured image, other exemplary methods and apparatus may use separate images of each eye. It is possible to determine contact lens size for each eye separately.

In exemplary methods and apparatus, the width of the captured image is typically about 4000 pixels and the centre of the eyes are typically about 3000 pixels apart. However, it is noted that the image may have a different width of any other amount of pixels. Typically, the distance between the centres of the eyes is approximately ¾ of the total width.

The eye detector 114 determines 202 the location of the eyes of the subject in the captured image. In exemplary methods and apparatus, the eye detector 114 uses reflections of the light source 111 to find the eyes of the subject in the captured image. Reflections of the light from the light source 111 can come from any shiny or moist object or part of the face of the subject. Typically, the captured image has a number of reflections, which may be caused by, for example, tears, the tip of the nose, teeth, earrings, sweat on the forehead etc.

The eye detector 114 is configured to determine the two reflections that identify the eyes of the subject. In exemplary methods and apparatus, this may be done by identifying all the reflections in the captured image and measuring, for each one, one or more parameters associated with that reflection. The measured parameters may be used to determine the reflections caused by the eyes. For example, the measured parameters may include the size, brightness (e.g., how close to full white it is), location and shape of the reflection.

A reflection from the eye of the subject is round, as it comes off of the cornea which has an outer surface that is very close to being part of the surface of a sphere. An exemplary reflection from an eye of the subject has a size typically in a range from 5 and 15 pixels across or, more specifically, in a range from 8 to 13 pixels across. A reflection from an eye of a subject has a difference between the horizontal and vertical measurements width measurement not more than 4 pixels or, more specifically, not more than 2 pixels. A reflection from an eye of a subject comprises not less than 80% or, more specifically, not less than 90% white pixels. In this regard, white pixels may comprise RGB values not less than 240, 240, 240. The eye detector 114 may be configured to detect one or more of the above features of reflections in the image.

The number of pixels identified above in relation to the parameters of the reflection from the eye is based on methods and apparatus using captured images having a total width of 4000 pixels and/or having a distance between the centre of the eyes of about 3000 pixels. If the captured image has a different total width in pixels, or a different distance between the centre of the eyes, the number of pixels representing the parameter may change accordingly and by the same or a similar ratio. In the exemplary methods and apparatus disclosed herein, a total captured image width of 4000 pixels is assumed but any measurement disclosed that is based on a number of pixels may be amended accordingly to be a the same or a similar ratio.

One or more identified reflections are assessed by the eye detector 114 against one or more of the criteria mentioned above and a shortlist is determined comprising those reflections that are possible reflections from the eye of the subject. Once the shortlist of possible eye reflections has been determined, each pair of reflections on the shortlist is given a score of how likely they are to be the correct pair, that is the pair of reflections from the eyes. This score is based on how close the distance between the pair of reflections is to a typical inter-pupillary distance (IPD) in humans (typically 50-65 mm), and how similar the two reflections are to each other. The reflections from eyes are far more symmetrical than an eye reflection would be to an earring/tear/sweat reflection and the distance between them must be a reasonable IPD.

The eye detector determines that the pair of reflections from the shortlist that achieved the highest score is the pair of eye reflections.

For each eye separately, the iris detector 116 finds 204 a rough estimate of the iris.

The iris is typically nearly round, with the horizontal diameter larger than the vertical diameter. The size of the iris is typically 10-15 mm across. It is typically visible on both sides and at the bottom, but often not visible at the top as it is obscured by the upper eyelid. It is surrounded by a whitish sclera on both sides and by skin tones above and below, often with eyelashes above. It has, somewhere near its centre, a pupil.

The iris is typically blue, green or brown, though its colour is often not the same throughout. A characteristic of all irises is that they are darker than their horizontal surroundings of the sclera.

Generally, the iris detector may determine the size and shape of the iris by fitting a plurality of ellipses to the captured image and selecting an iris ellipse having a best fit to the iris to be representative of the size and shape of the iris. The best fit ellipse may be determined as the ellipse having the fewest skin pixels and/or sclera pixels inside, and/or having the fewest iris pixels outside. A specific example is given below. Further, it is noted that circles are considered to be a subset of ellipses.

The iris detector 116 may be configured to convert the RGB values of the pixels of the eye in the captured image to a luma value, which is a single value for each pixel that roughly represents how bright that pixel is. Iris pixels are those having a luma value below an iris threshold value. Sclera pixels are those having a luma value above a sclera threshold value. Typical luma values are up to 100 for the iris and over 150 for the sclera.

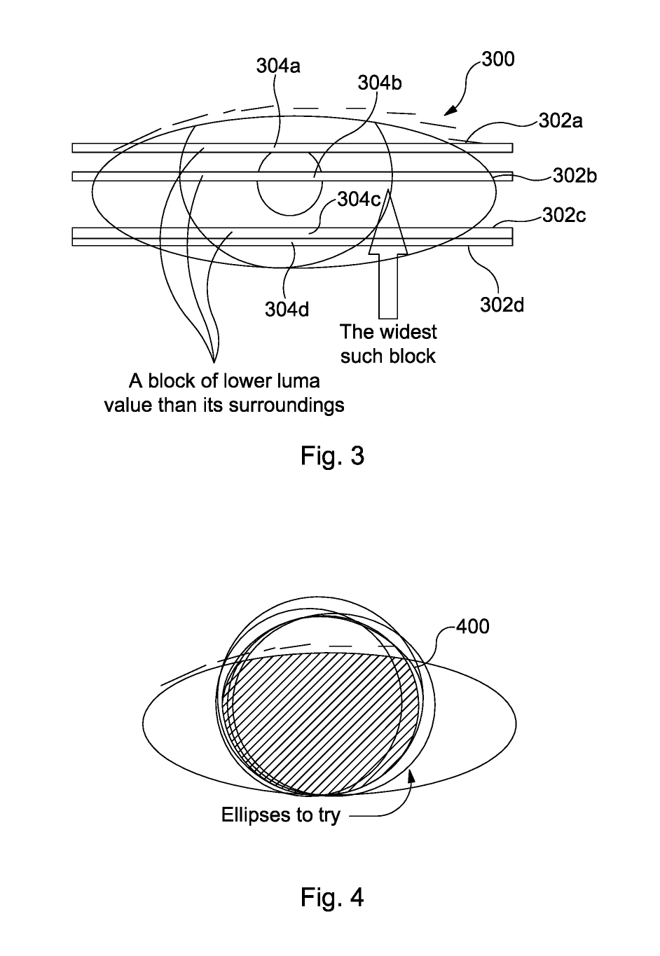

The iris detector 116 may determine a rough centre for the iris. This may be done by superimposing on the eye region 300 of the captured image a plurality of horizontal strips 302a-d. One exemplary configuration of strips is shown in FIG. 3. However, other configurations are possible, including that the eye area 300 is divided into a plurality of strips covering the entire area. The strips 300a-d may be the same or different sizes. In each strip 302a-d the distance between the limits of the iris pixels is determined and the maximum distance is between the horizontal maxima of the iris pixels. The distance is between where the iris pixels begin and end within a strip 302a-d. This is shown as the shaded areas 304a-d in FIG. 3. The strip 302a-d with the largest such distance is determined to be approximately coincident with a centre line through the iris. This in turn allows the determination of a rough centre of the iris being the centre of the measured distance and a rough width of the iris being the distance itself.

The iris detector 116 may determine a rough estimate for the iris. The rough estimate for the iris may be determined to be an iris circle around the approximate centre having a diameter equal to the measured distance.

The iris detector 116 may then determine 206 an accurate location for the iris in the captured image. This may be done by classifying all pixels within the circle defining the rough iris location into categories using their luma value, saturation value and a third value that defines the “redness” of the pixel. As used herein, the term “redness” encompasses the greater of R minus G and R minus B of a pixel, wherein R is red, G is green and B is blue. The redness roughly gives a measure of which pixels have a colour that is influenced by red blood behind them (such as skin and sclera regions of the eye) and which pixels have a colour that is more influenced only by their own pigmentation (such as the iris) or have no colouring at all (like the pupil).

Based on the luma, saturation and redness value for each pixel, the pixels of the rough iris may be separated into three categories:
“Pink”—relatively high redness and luma, intermediate saturation. Typically skin. (Note that skin shades are dealt with effectively here. All skin, from the lightest to the darkest, falls into this category). Typical values for “pink” pixels are redness (R) value over 150, luma value over 120 and a saturation value in a range from around 30-80.
“White”—relatively high luma, low saturation and redness. Typically sclera. Typical values for “white” pixels are luma over 150, saturation under 50 and R over 130.
“Other”—this typically includes the iris and pupil.

The boundaries of these categories may be determined dynamically from the actual values found for the pixels in the captured image of the eye in a way that will maximise the separation into categories. That is, if different values for the boundaries between pixel categories are tried, different separation between pixel categories result, i.e. pixels of the image are categorised differently. For example, if the boundaries are set too high, all the pixels will fall into the “other” category. Therefore, in exemplary methods and apparatus, different boundaries are tried until a separation into three roughly equal-sized categories is reached. This is because the areas of the sclera, the iris plus the pupil, and of the skin directly adjacent to the eye are of—very roughly—the same size.

Note that pixels relating to eyelashes and other features in the eye region may fall into any of the categories. This does not matter as they are typically few.

The iris detector 116 may then superimpose a plurality of ellipses on the eye region of the captured image approximately centred on the rough centre of the iris. In exemplary methods and apparatus, thousands of ellipses may be superimposed on the image. Each ellipse may have a different size and shape and may be differently positioned over the rough iris. Each ellipse may have a centre within 20 pixels, horizontally and/or vertically, of the centre of the rough iris, a width-to-height ratio in a range from 0.9 to 1.0, and a horizontal diameter in a range from 20 pixels less than that of the rough iris to 20 pixels more. FIG. 4 shows a plurality of ellipses around the rough iris and an iris ellipse 400 is considered to be the best fit based on a score as set out below.

SCORE=NWO−4*NWI+NOI−NOO

Where NWI is the number of “white” pixels inside the ellipse, NWO is the number of “white” pixels outside the ellipse, NOI is the number of “other” pixels inside the ellipse and NOO is the number of “other” pixels outside the ellipse. The score is determined based on the fact that the best fit ellipse will have “pink”, skin, pixels either inside or outside the ellipse with the highest “other”, iris or pupil, pixels inside the ellipse and the highest number of “white”, sclera, pixels outside the ellipse.

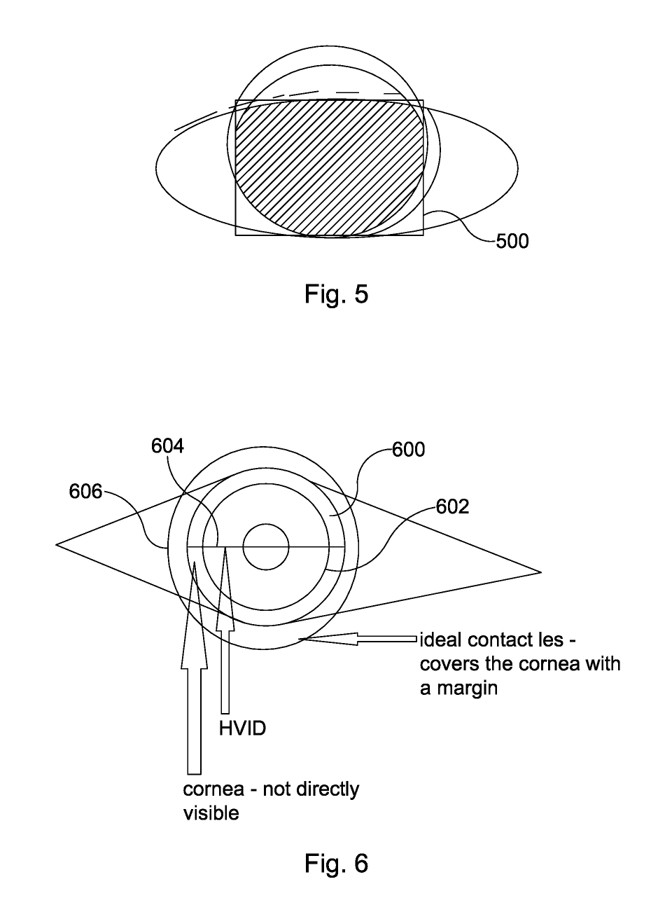

Based on the best fit ellipse, the iris detector 116 determines a bounding box 500 that has sides at the limits of the “other” pixels inside this ellipse, as shown in FIG. 5. The iris detector 116 determines that the part of the ellipse bounded by this box is the accurate location of iris.

Once the accurate iris has been determined, the HVID can be calculated and is the horizontal diameter of the best fit ellipse. In addition, a vertical iris diameter and/or any other iris diameter, such as a diagonal iris diameter, may be determined. As the best fit ellipse is determined, this allows the iris detector 116 to estimate the size and shape of the iris in the region that is covered by the eye lid. This would not be possible using manual techniques or techniques that simply measure the diameter of the iris directly.

As can be seen in FIG. 6, the iris 600 is larger in diameter than the cornea 602, which is not directly visible in the captured image. Therefore, the size of the cornea 602 can be determined based on the HVID 604. A contact lens 606 is also shown in FIG. 6 and is larger in diameter than the cornea 602 and the iris 600.

The cornea determiner 118 determines 208 the size of the cornea of the subject based on the determined iris size. In exemplary methods and apparatus, the cornea determiner 118 determines that the diameter of the cornea is in range from 0.3 mm to 0.5 mm or, more specifically, 0.4 mm smaller than the HVID determined from the iris. The cornea is assumed to be a circle, although in reality it is actually more like a squashed circle, an ellipse. The circle is assumed to be concentric with the best fit ellipse of the iris and having a diameter less than the HVID by the amounts mentioned above is determined to be the location and size of the cornea.

Based on the size of the cornea, the contact lens determiner 120 determines 210 one or more parameters of contact lens suitable for the subject. In exemplary methods and apparatus, one or more physical parameters are determined. For example, a diameter of the contact lens may be determined to be larger in diameter than the diameter of the cornea by a distance in a range from 1 mm to 3 mm, or in specific exemplary arrangements 2 mm.

In exemplary methods and apparatus, the contact lens determiner 120 may determine that an off the shelf contact lens is suitable for the subject based on the determined cornea size. Off the shelf contact lenses have one of a plurality of set diameters and the contact lens determiner 120 may determine that one of those contact lens diameters is suitable for the subject if it falls within a range either side of the determined contact lens size.

In other exemplary methods and apparatus, the contact lens determiner 120 may determine one or more physical parameters of a contact lens that may be used to manufacture contact lenses that are made to order. Made to order contact lenses can result in a better fitting contact lens for a subject who might find the off the shelf contact lenses uncomfortable or inappropriate.

One or more features of the eye comprising HVID, pupil size, pupil eccentricity and corneal curvature may be determined.

FIG. 2 shows three exemplary types of contact lens that may be determined: non-uniform, in which the optical features are non-uniform; off the shelf; and made to order. Each of these contact lens types are determined based on further measured eye features in addition to the diameter of the cornea. However, it should be understood that these are exemplary only and the methods and apparatus disclosed herein may be configured to determine any physical parameter and/or optical parameter of a contact lens based on one or more of: the cornea diameter; the lid margins; the pupil eccentricity; the pupil diameter; and the corneal curvature.

Off the shelf contact lenses are those that have a number of fixed physical parameters. The most common off the shelf contact lenses have uniform optical features. However, off the shelf contact lenses may also have non-uniform optical features. That is, non-uniform contact lenses may be either off the shelf contact lenses or made to order contact lenses. In addition, custom made contact lenses may have uniform or non-uniform optical features. One or more steps of the exemplary routes in FIG. 2 may be combined with steps of another exemplary route.

In the exemplary method for determining a parameter of a made to order contact lens, a pupil detector 122 is configured to determine 212 the diameter and/or location of the pupil of each eye of the subject in the captured image. Although made to order contact lenses may be manufactured using the HVID alone, exemplary methods and apparatus may determine one or more further parameters such as pupil size and/or corneal curvature.

Generally, the pupil detector 122 may be configured to fit a plurality of ellipses to the captured image and selecting a pupil ellipse that has the best fit to the pupil to be representative of the diameter of the pupil. The best fit ellipse may be determined as the ellipse having the fewest non-pupil pixels inside and/or the fewest pupil pixels outside. A specific example is given below.

Taking one iris at a time, the pixels in the best fit ellipse are classified by their luma value. The pupil is darker than the iris and usually nearly black. Therefore, the pixels inside the best fit ellipse are separated into two categories, “dark” (pupil) and “light” (iris), according to this classification. Typically, dark pupils may have a luma value under 30 and light pupils may have a luma value of 30 or more. Dark pixels are those having a luma less than a pupil threshold value and light pixels are those having a luma greater than an iris threshold value.

A boundary between the dark and light categories is determined dynamically from the actual luma values of the pixels inside the best fit ellipse for a given eye in a way that will maximise the separation into the two categories, as discussed above. In this case, the best boundary is such that about ⅓ of pixels fall into the pupil category and about ⅔ fall into the iris category, reflecting the typical proportions of a pupil within the iris in normal lighting conditions.

A plurality of circles is superimposed on the image based on the location of the centre of the iris and each circle is assessed to determine. In exemplary methods and apparatus, thousands of circles may be superimposed. Each circle may have a centre within 80 pixels, horizontally and/or vertically, of the centre of the iris, and a diameter in a range from ⅛ to ⅔ of the horizontal diameter of the iris.

Each circle receives a score which is determined by:

SCORE=PDI−PDO

PDI relates to the number of dark pixels inside the circle. PDI may be the proportion of dark pixels inside the circle (a number from 0 to 1) and is calculated as the number of dark pixels in the circle divided by the area of the circle in pixels. PDO relates to the number of dark pixels outside the circle. PDO may be the proportion of dark pixels outside the circle. This is calculated as the number of dark pixels outside the circle divided by the area of the circle, which can be greater than 1. The circle with the highest score is determined to be the size and location of the pupil.

If there is no circle with a score of over 0.5, an alternative method for finding the pupil may be undertaken. In particular, due to the red-eye effect in photographs, the pupil may appear red rather than black in the captured image. This red can vary from bright orange to dark purple. When the pupil is thus lightened, and the iris is relatively dark (such as in grey- or brown-eyed people), the method mentioned above may fail because the luma value of pupil pixels is not lower than that of iris pixels.

In such circumstances, the pupil may be determined again but instead of using luma to classify the pixels and score each circle, a measure of “brightness ignoring red” (BIR) is used, which is defined as:

BIR=MAX(G,B)

Thus, BIR is high for any iris. A blue iris will typically have a BIR of over 100, and a brown one of about 60. For the pupil, even if flooded with red, the BIR is lower.

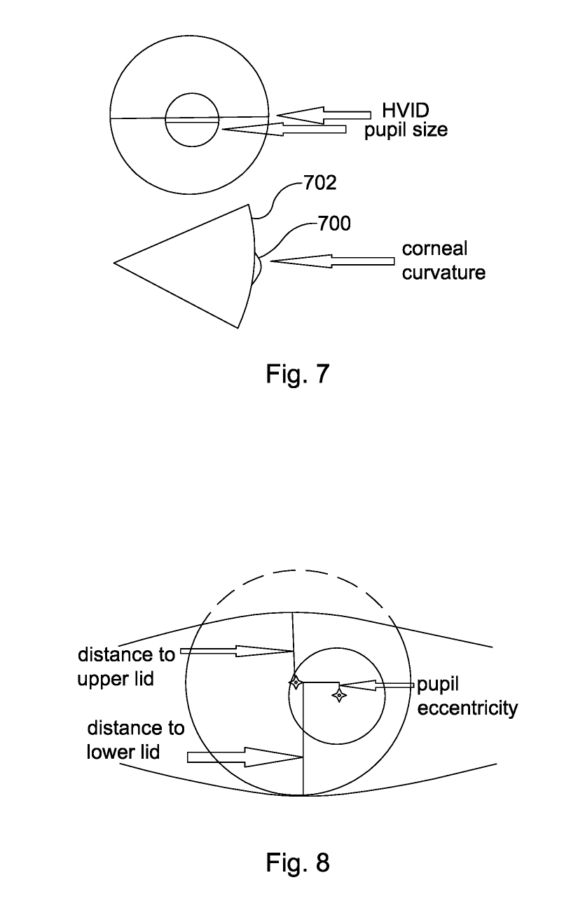

Referring to FIG. 7, the corneal curvature is the distance that the central part 700 of the cornea—typically the central 3 mm—“sticks out” of the roughly spherical shape of the eye 702.

The curvature detector 124 determines 214 the corneal curvature of the eye. The contact lens determiner 120 may then determine one or more physical parameters, such as the base curve, of a contact lens based on one or more of the pupil diameter and the corneal curvature. In exemplary methods and apparatus, the corneal curvature may be measured by a separate device and input to the apparatus.

A growing sub-specialty in contact lenses is the field of non-uniform contact lenses, such as toric, bifocal or multifocal contact lenses, which are similar to bifocal or multifocal glasses. As contact lens users age, they often do not want to give up the use of contact lenses and would like to try bifocal or multifocal lenses rather than add glasses on top of existing contact lenses or instead of them.

The success of non-uniform contact lenses depends to a much greater extent than that of standard contact lenses on the detailed measurement of features of the eye. These measurements are typically the iris and pupil sizes, as above, but may also include one or more of the distance from the centre of the pupil to the upper and/or lower lid margins and the eccentricity of the pupil, which is the horizontal and vertical distance between the pupil centre and the iris centre.

If non-uniform contact lenses are required, the lid margin detector 126 may determine 216 the location of the lid margins. This may be done based on the accurate iris determined earlier and defined using a bounding rectangle of “other” (pupil and iris, as opposed to sclera or skin) pixels. The horizontal boundaries of this rectangle were used to define the HVID—the horizontal visible iris diameter. This is because the iris is typically fully visible, horizontally, with sclera on both sides. The vertical boundaries, however, are not the vertical size of the iris. In a typical situation, the iris is not fully visible vertically. The bottom often is (see FIG. 3 to illustrate this) but the top is, in nearly all cases, cut off by the upper eyelid. The distance from the centre of the pupil to these top and bottom visibility cut-off points is significant for contact lens fitting and for aid in diagnosis of neurological and other conditions, which often cause the upper eyelid to “droop”—that is, to hide more of the top part of the iris and pupil than in the general population. The centre of the pupil may be determined as set out above and the distance from the centre of the pupil to the lid margins may be calculated as the distance from the centre to the top and bottom of the bounding rectangle. It is presented as two numbers for each eye—for example, 2.34 mm from pupil centre to top lid margin and 6.13 mm from pupil centre to bottom lid margin.

The eccentricity detector 128 determines 218 the pupil eccentricity. Typically, the pupil and the iris are not concentric. The pupil is, in most cases, nasally displaced (that is, shifted towards the centre of the face) by a small amount—about 0.4 mm on average in adults. It may also be shifted up or down relative to the iris, often by a smaller amount. These shifts—the “pupil eccentricity”—are interesting for the fitting of multifocal contact lenses as they affect where the visual axis is located relative to the cornea (and therefore, relative to the contact lens). The eccentricity is calculated as two distances—horizontal and vertical—between the centre of the iris and the centre of the pupil, both of which may be determined as set out above. It is presented as two numbers for each eye—for example, 0.46 mm nasal and 0.21 mm up.

The pupil eccentricity may be used to determine a displacement of the optical features of the contact lens from a centre of the contact lens. In exemplary methods and apparatus, the optical features may be displaced from the centre of the contact lens by an amount corresponding to the pupil eccentricity. In such contact lenses, it may be necessary to provide a means for orienting the contact lens with respect to the eye.

In exemplary methods and apparatus, the parameters of the eye (e.g., cornea size, pupil size and/or corneal curvature) are calculated for each eye separately and used to determine contact lens parameters for each eye. In other exemplary methods and apparatus, an image may comprise only one eye of a subject, in which case the parameters of the eye are determined for that eye only.

A computer program may be configured to provide any of the above described methods. The computer program may be provided on a computer readable medium. The computer program may be a computer program product. The product may comprise a non-transitory computer usable storage medium. The computer program product may have computer-readable program code embodied in the medium configured to perform the method. The computer program product may be configured to cause at least one processor to perform some or all of the method.

Various methods and apparatus are described herein with reference to block diagrams or flowchart illustrations of computer-implemented methods, apparatus (systems and/or devices) and/or computer program products. It is understood that a block of the block diagrams and/or flowchart illustrations, and combinations of blocks in the block diagrams and/or flowchart illustrations, can be implemented by computer program instructions that are performed by one or more computer circuits. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and/or flowchart block or blocks, and thereby create means (functionality) and/or structure for implementing the functions/acts specified in the block diagrams and/or flowchart block(s).

Computer program instructions may also be stored in a computer-readable medium that can direct a computer or other programmable data processing apparatus to function in a particular manner, such that the instructions stored in the computer-readable medium produce an article of manufacture including instructions which implement the functions/acts specified in the block diagrams and/or flowchart block or blocks.

A tangible, non-transitory computer-readable medium may include an electronic, magnetic, optical, electromagnetic, or semiconductor data storage system, apparatus, or device. More specific examples of the computer-readable medium would include the following: a portable computer diskette, a random access memory (RAM) circuit, a read-only memory (ROM) circuit, an erasable programmable read-only memory (EPROM or Flash memory) circuit, a portable compact disc read-only memory (CD-ROM), and a portable digital video disc read-only memory (DVD/Blu-ray).

The computer program instructions may also be loaded onto a computer and/or other programmable data processing apparatus to cause a series of operational steps to be performed on the computer and/or other programmable apparatus to produce a computer-implemented process such that the instructions which execute on the computer or other programmable apparatus provide steps for implementing the functions/acts specified in the block diagrams and/or flowchart block or blocks.

Accordingly, the invention may be embodied in hardware and/or in software (including firmware, resident software, micro-code, etc.) that runs on a processor, which may collectively be referred to as “circuitry,” “a module” or variants thereof.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Finally, other blocks may be added/inserted between the blocks that are illustrated.

The skilled person will be able to envisage other embodiments without departing from the scope of the appended claims.

1. An apparatus for processing an image to determine one or more parameters of a contact lens suitable for a human or animal subject, the image comprising a plurality of pixels forming an image of at least part of one eye of the subject, the apparatus comprising:
an iris detector configured to determine a diameter of an iris of the subject;
a cornea determiner configured to determine a diameter of a cornea of the subject based on the determined diameter of the iris; and
a contact lens determiner configured to determine the one or more parameters of a contact lens for the subject based on the determined cornea diameter.

2. An apparatus according to claim 1, further comprising a camera configured to capture the image and a light source associated with the camera.

3. (canceled)

4. An apparatus according to claim 1, further comprising an eye detector configured to locate one or more eyes of the subject in the image by identifying in the image reflections of a light source used when the image was captured, based on the RGB values of pixels of the image, and to identify which of the reflections is from the at least one eye of the subject.

5. (canceled)

6. An apparatus according to claim 1, wherein the iris detector is configured to determine the diameter of the iris by determining an iris ellipse having a best fit to the iris of the subject.

7. An apparatus according to claim 6, wherein the iris detector is configured to determine iris pixels of the image based on a luma value of each pixel in an eye region, and to determine the iris ellipse as an ellipse of a plurality of ellipses having the most iris pixels inside.

8. (canceled)

9. An apparatus according to claim 6, wherein the iris detector is configured to determine the iris ellipse based on one or more of a luma value for iris pixels, a saturation value for the iris pixels and a redness value for the iris pixels.

10. (canceled)

11. An apparatus according to claim 6, wherein the iris detector is configured to classify pixels proximal to the iris as “pink”, “white” or “other” and to determine a score for each of a plurality of possible iris ellipses based on:
NWO−4*NWI+NOI−NOO
where NWI is the number of white pixels inside the ellipse, NWO is the number of white pixels outside the ellipse, NOI is the number of other pixels inside the ellipse and NOO is the number of other pixels outside the ellipse,
and wherein the iris detector is further configured to determine the iris ellipse as the ellipse having the highest score.

12. An apparatus according to claim 6, wherein the iris detector is configured to determine a horizontal visible iris diameter, HVID, as the horizontal diameter of the iris ellipse.

13. An apparatus according to claim 6, wherein the iris detector is configured to determine one or more of: a vertical iris diameter; and a diagonal iris diameter, based on the iris ellipse.

14. An apparatus according to claim 13, wherein the contact lens determiner is configured to determine the one or more parameters of the contact lens based on the vertical iris diameter and/or the diagonal iris diameter.

15. (canceled)

16. An apparatus according to claim 1, wherein the iris detector is configured to determine an approximate centre of the iris by determining an approximate horizontal centre line of the iris by determining horizontal maxima of the iris pixels.

17. (canceled)

18. (canceled)

19. An apparatus according to claim 1, further comprising a pupil detector configured to determine a diameter and/or an eccentricity of a pupil of the subject,
and wherein the contact lens determiner is configured to determine the one or more parameters of the contact lens based on the determined pupil diameter and/or pupil eccentricity.

20. An apparatus according to claim 19, wherein the contact lens determiner is configured to determine a location of one or more optical features of a contact lens based on a determined pupil eccentricity.

21. An apparatus according to claim 19, wherein the pupil detector is configured to determine the diameter of the pupil by determining a pupil ellipse that has a best fit with the pupil of the subject.

22. An apparatus according to claim 21, wherein the pupil ellipse is determined based on a luma value and/or a brightness ignoring red, BIR, value of pixels in the eye region.

23. An apparatus according to claim 22, wherein the pupil detector is configured to classify the pixels in the eye region as pupil pixels having a luma value less than a threshold value and iris pixels having a luma value greater than a first iris threshold value, or to classify the pixels in the eye region as pupil pixels having a BIR value less than a second pupil threshold value and iris pixels having a BIR value greater than a second iris threshold value, the pupil detector being further configured to determine a score for each of a plurality of possible pupil ellipses based on:
PDI−PDO
where PDI is the proportion of pupil pixels inside the ellipse and PDO is the proportion of pupil pixels outside the ellipse,
and wherein the pupil detector is further configured to determine the pupil ellipse as the ellipse having the highest score.

24. (canceled)

25. (canceled)

26. An apparatus according to claim 19, wherein the pupil detector is configured to determine a centre of the pupil, the apparatus further comprising a lid margin detector configured to determine a distance from the centre of the pupil to one or more eyelids of the subject.

27. An apparatus according to claim 1 wherein the contact lens determiner is configured to determine one or more optical parameters of the contact lens based on the determined distance from the centre of the pupil to one or more eyelids,
and wherein the optical parameter comprises a location of optical features on the contact lens.

28. A method for processing an image to determine one or more parameters of a contact lens suitable for a human or animal subject, the image comprising a plurality of pixels forming an image of at least part of one eye of the subject, the method comprising:
determining, by an iris detector, a diameter of an iris of the subject;
determining, by a cornea determiner, a diameter of a cornea of the subject based on the determined diameter of the iris; and
determining, by a contact lens determiner, the one or more parameters of a contact lens for the subject based on the determined cornea diameter.

29. A computer program comprising instructions which, when executed on at least one processor, cause the at least one processor to carry out the method according to claim 28.

30. A carrier containing the computer program of claim 29, wherein the carrier is one of an electronic signal, optical signal, radio signal, or non-transitory computer readable storage medium.
